(19) United States
(12) Patent Application Publication
(10) Pub. No.: US 2023/0355693 A1
(43) Pub. Date: Nov. 9, 2023

(54) METHODS FOR DRYING AND STERILIZATION OF CANNABIS MATERIAL

(71) Applicant: BUZZELET DEVELOPMENT AND TECHNOLOGIES LTD., OR-AKIVA (IL)

(72) Inventors: AHARON EYAL, JERUSALEM (IL); MEIR ARIEL, KATZIR (IL)

(21) Appl. No.: 17/919,753

(22) PCT Filed: Apr. 19, 2020
(86) PCT No.: PCT/IB2020/053697

§ 371 Date: Oct. 19, 2022

Related U.S. Application Data
(60) Provisional application No. 62/950,131, filed on Dec. 19, 2019.
(60) Provisional application No. 62/836,678, filed on Apr. 21, 2019.

(51) Int. Cl. A61K 36/185; A24B 15/18; A24B 15/22

(52) U.S. Cl. CPC A61K 36/185; A24B 15/183; A24B 15/22; A61K 2236/17; A61K 2236/13; A61K 2236/11

(57) ABSTRACT

Provided are methods for drying and sterilizing cannabis plant material comprising providing feed cannabis plant material having a water content of M0, wherein M0 is greater than 45%; partially drying the feed cannabis plant material to form a partially dried cannabis plant material having a water content of M1, wherein M1 is in the range of between 12% wt and 40% wt; sterilizing the partially dried cannabis plant material to form sterilized partially dried cannabis plant material having a water content of M3; and when said M3 is less than 8% wt or greater than 16% wt, adjusting said M3 to provide a water content of M2, wherein said M2 is between 8% wt and 16% wt. Further provided are methods and systems for drying of cannabis plant material.

### FIELD OF THE INVENTION

The field of art to which this invention generally pertains is methods for drying of cannabis plant material, and more specifically to methods for partially drying and sterilizing cannabis plant material, as well as to systems for drying cannabis plant material.

### BACKGROUND OF THE INVENTION

Material obtained from the cannabis plant can be used for many medicinal or therapeutic applications.

It is necessary to dry the cannabis plant material prior to consumption or storage.

It is further necessary to sterilize the cannabis plant material prior to consumption or storage. Prior art sterilization methods typically involve use of beta- or gamma-irradiation and cannot be conducted at the post-harvest facility. The harvested cannabis plant material is therefore fully dried, packaged and sent to a second site for sterilization, wherein sterilization of the cannabis plant material is carried out on the material while within the package. In some cases, the dried cannabis plant material is packaged at the drying site into packages of a size intended for sale, such as 10 g packages, sterilized within these packages and sent for sale as such. Alternatively, in some cases, the dried cannabis plant material is packaged in larger packages than those intended for sale, such as 1 kg packages, sterilized within these packages and subsequently divided into smaller packages for sale.

Such two-site operations require complex logistics, including transportation of the cannabis plant material. Additionally, during sterilization, loss of active components occurs, possibly due to the fact that the plant material is quite dry and is therefore susceptible to heating.

There exists a need for improved methods of drying, and optionally sterilizing, material obtained from the cannabis plant which overcome at least some of the limitations of the prior art.

### SUMMARY OF THE INVENTION

According to an aspect of some embodiments of the present invention, there is provided a method for drying and sterilizing of cannabis plant material, the method comprising:
(i) providing feed cannabis plant material having a water content of M0, wherein M0 is greater than 45%;
(ii) partially drying said feed cannabis plant material to form a partially dried cannabis plant material having a water content of M1, wherein M1 is in the range of between 12% wt and 40% wt;
(iii) sterilizing said partially dried cannabis plant material to form sterilized partially dried cannabis plant material having a water content of M3; and
(iv) when said M3 is less than 8% wt or greater than 16% wt, adjusting said M3 to provide a water content of M2, wherein said M2 is between 8% wt and 16% wt.

According to a further aspect of some embodiments of the invention, there is provided a method for drying of cannabis plant material, the method comprising:
(i) providing a system comprising at least one primary drying chamber, which at least one primary drying chamber has a volume V1, and in operation provides a relative humidity RH1 and a temperature T1; and at least one secondary drying chamber which at least one secondary drying chamber has a volume V2, and in operation provides a relative humidity RH2 and a temperature T2, and wherein said system comprises at least one selected from the group consisting of
   (a) said V2 being greater than said V1;
   (b) said RH2 being lower than said RH1; and
   (c) said T2 being higher than said T1;
(ii) providing a feed cannabis plant material having a water content of M0 percent and a relative water load L0 wherein L0 equals M0/(100−M0) and wherein M0 is greater than 45%;
(iii) keeping said feed cannabis plant material in said at least one primary drying chamber for a duration of D1 days, during at least a portion of which said duration of D1 days said primary drying chamber is in operation, whereby said water content M₀ of said feed cannabis plant material drops to form a partially dried cannabis plant material having a water content of M1 percent and a relative water load L1 wherein L1 equals M1/(100−M1);
(iv) transferring said partially dried cannabis plant material into said at least one secondary drying chamber and keeping it there for a duration of D2 days, during at least a portion of which said duration of D2 days said drying chamber is in operation, whereby said water content M1 of said partially dried cannabis plant material drops to form a mostly dried cannabis plant material with water content of M2 percent, and a relative water load L2 wherein L2 equals M2/(100−M2) and wherein said M2 is less than 20%: and optionally
(v) maintaining said mostly dried cannabis plant material for at least 3 days at relative humidity lower than said RH1,
   wherein said T1, said T2, said RH1, said RH2, said D1 and said D2 are selected so that (L0−L1)/(L1−L2) is greater than 2.

According to a further aspect of some embodiments of the present invention, there is provided a system for drying of cannabis plant material, the system comprising:
(i) at least one primary drying chamber, having a volume V1, and configured to provide, during operation, a relative humidity RH1 and temperature T1; and
(ii) at least one secondary drying chamber, having a volume V2, and configured to provide, during operation, a relative humidity RH2 and temperature T2,
wherein said system is characterized by at least one selected from the group consisting of (a) said V2 being greater than said V1; (b) said RH2 being lower than said RH1; and (c) said T2 being higher than said T1.

### DETAILED DESCRIPTION OF THE INVENTION

The present invention relates to methods for partially drying and sterilizing cannabis plant material, as well as to systems for drying cannabis plant material.

Prior art methods for drying and sterilizing cannabis plant material typically dry the plant material prior to sterilization to a final moisture content of about 10% wt. In such methods, sterilization of the dry plant material result in significant loss of active material. Furthermore, since sterilization is conducted within a package, relative moisture may increase due to heating which occurs as a consequence of the sterilization process, which may lead to fungal re-growth.

The present Inventors have surprisingly found that by only partially drying the cannabis plant material to a preferred water content of no less than 12% wt prior to sterilization, which is higher than the final required level, at least some of the disadvantages associated with prior art drying and sterilization methods are overcome. The water content may then be adjusted to a desired final level post-sterilization, by either further drying or adding moisture, as required.

The methods of the present invention enable both drying and sterilization of cannabis plant material to be conducted at a single site, optionally but not necessarily, in a single system comprising drying and sterilizing components. Conducting drying and sterilization at a single site obviates the need for packaging the dried cannabis plant material for transportation, and the associated disadvantages thereof.

Furthermore, when using a typical drying protocol, the cannabis plant material remains unsterilized during the whole drying (and curing) process, which typically takes 2-4 weeks such that a large volume of the drying facility is potentially contaminated and is considered a quarantine area. The drying cannabis plant material therefore cannot be kept in the same room with other batches. Drying to a water content of about 25% is rapid, normally occurring within 3 days, while further drying takes more than 2 weeks. Since sterilized cannabis plant material can be liberated from the quarantine area, the quarantine time for the method of the present invention is decreased by about 80% as compared to that of prior art methods.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for describing particular embodiments only and is not intended to be limiting of the invention.

Unless otherwise defined, all technical and scientific terms used herein have the same meaning as commonly understood by one of ordinary skill in the art to which this invention belongs.

As used herein, the term “feed cannabis plant material” refers to a cannabis plant material as obtained from a cannabis plant as harvested, prior to subjecting such material to a sterilizing step and while water content is greater than 45%.

Unless indicated otherwise, the term “cannabis plant material” as used herein refers to a material obtained from a plant of the species Cannabis Sativa.

As used herein the term “sterilizing” refers to a treatment for the reduction of microbial contamination, e.g. as in pasteurizing.

As used herein, the term “single processing site” is intended to refer to a location at which both a drying apparatus and a sterilizing apparatus are provided at a distance of no more than about 1 km one from the other, preferably no more than 100 m from each other. Preferably, sterilization at the single processing site is carried out no more than 2 hours after completion of the partial drying process.

As used in the description of the invention and the appended claims, the singular forms “a,” “an,” and “the” are intended to include the plural forms as well, unless the context clearly indicates otherwise.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

Unless indicated otherwise, percent is weight percent and ratio is weight/weight ratio.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for drying and sterilizing of cannabis plant material, the method comprising:
(i) providing feed cannabis plant material having a water content of M0, wherein M0 is greater than 45%;
(ii) partially drying said feed cannabis plant material to form a partially dried cannabis plant material having a water content of M1, wherein M1 is in the range of between 12% wt and 40% wt;
(iii) sterilizing said partially dried cannabis plant material to form sterilized partially dried cannabis plant material having a water content of M3; and
(iv) when said M3 is less than 8% wt or greater than 16% wt, adjusting said M3 to provide a water content of M2, wherein said M2 is between 8% wt and 16% wt.

According to an embodiment, said feed cannabis plant material is green and the method is applied to the material within zero to 24 hours from harvest, such as substantially immediately after harvest, within 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or 24 hours from harvest.

At the time of harvest, the cannabis plant material has a relatively high water content (typically in the range of about 70-80%) which decreases rapidly following harvesting. Hence, according to an embodiment, the water content of feed cannabis plant material is greater than 45% wt, greater than 50% wt, greater than 60% wt, greater than 70% wt or greater than 80% wt.

Water content may be determined by any method known in the art. According to a preferred embodiment, water content is determined by the Loss-on-Drying Test e.g. according to ASTM E1868. As used herein, the term “water content” is intended to mean the loss on drying as determined according to ASTM E1868.

According to an embodiment, drying of the feed cannabis plant material is carried out as a continuous process.

According to an alternative embodiment, drying is carried out in batches. According to some embodiments, the batches for drying each have a weight of from about 1 kg to about 2000 kg, such as about 1 kg, about 5 kg, about 10 kg, about 15 kg, about 20 kg, about 25 kg, about 30 kg, about 35 kg, about 40 kg, about 45 kg, about 50 kg, about 100 kg, about 200 kg, about 300 kg, about 400 kg, about 500 kg, about 600 kg, about 700 kg, about 800 kg, about 900 kg, about 1000 kg, about 1500 kg or about 2000 kg.

Drying may be carried out by any method known in the art. Selection of suitable drying conditions, such as relative humidity, air flow and temperature would be a matter of routine procedure for one of ordinary skill in the art. In some embodiments, drying is carried out at a temperature of no more than 40° C., such as 10° C.-40° C., 15° C.-35° C. or 20° C.-30° C. for a duration of 0.5-5 days and at a relative humidity of 30-80%, 35-70% or 40-60%.

According to some preferred embodiments, drying to a water content of about 25% is carried out over a period of about 3 days, while further drying to about 12% is carried out over a further period of 1-3 weeks.

It is well known that accelerated drying impedes the quality of the dried cannabis material. Without wishing to be limited by theory, the present inventors hypothesize that during the drying process, several chemical and biological changes take place, each of which requires a particular duration of time in which to achieve completion. It is therefore considered highly likely that the rate and extent of such reactions is dependent on the water content of the plant material at each step of the process. It is also considered likely that temperature impacts reaction rates. Furthermore, while some of those reaction steps may take place simultaneously, others might be sequential, such that one such reaction step produces an intermediate reagent for the following one. Hence the duration of drying, moisture content in each step and temperature at each step are important parameters in generating properly dried cannabis plant material. Temperature also affects the drying rate and duration, as does the relative humidity of the drying environment.

Some preferred conditions are disclosed in the following. However, it should be kept in mind that preferred drying conditions may change according to the properties of the feed cannabis plant material used, e.g. inflorescence size and composition (which may vary from one strain to another) and time from harvest.

According to an embodiment, feed cannabis plant material comprises cannabis inflorescence (flowers). According to an embodiment, said feed cannabis plant material comprises ground cannabis inflorescence. According to an embodiment, said feed cannabis plant material comprises industrial hemp.

According to an embodiment, partially drying said feed cannabis plant material is carried out in a chamber having controlled temperature and controlled relative humidity (i.e. relative humidity of 30-80%) selected according to the property of the plant material to be partially dried to reach the desired moisture.

According to an embodiment, M1 is about 12% wt, 15% wt, 20% wt, 25% wt, 30% wt, 35% wt or 40% wt.

According to an embodiment, partially drying said feed cannabis plant material is carried out by heating at a temperature in the range of from about 10° C. to about 60° C., such as about 10° C., about 15° C., about 20° C., about 25° C., about 30° C., about 35° C., about 40° C., about 45° C., about 50° C., about 55° C. or about 60° C.

According to an embodiment, partially drying said feed cannabis plant material is carried out for a duration of between 0.5 and 5 days, such as 0.5 days, 1 day, 1.5 days, 2 days, 2.5 days, 3 days, 3.5 days, 4 days, 4.5 days or 5 days.

According to an embodiment, said sterilizing said partially dried cannabis plant material is carried out by at least one of gamma irradiation, beta irradiation, ultraviolet irradiation, microwave irradiation, radio-frequency irradiation, a contact with an oxidant and plasma treatment.

According to an embodiment said sterilizing said partially dried cannabis plant material is carried out by radio-frequency irradiation. According to one such embodiment, sterilizing by radio-frequency irradiation brings the temperature of said plant material to a temperature in the range of from about 60° C. to about 90° C., from about 60° C. to about 85° C., from about 65° C. to about 85° C., or from about 70° C. to about 83° C., such as about 70° C., about 71° C., about 72° C., about 73° C., about 74° C., about 75° C., about 76° C., about 77° C., about 78° C., about 79° C., about 80° C., about 81° C., about 82° C. or about 83° C.

According to a preferred embodiment, sterilization by radio-frequency irradiation is carried out on batches each having a weight of from about 8 kg to about 20 kg. Such an embodiment provides accurate temperature control for a precise duration, enabling optimal sterilization conditions thereby resulting in minimal loss of active materials during the sterilization process.

According to some embodiments wherein sterilization is carried out in batches, the sterilization duration is about 15 minutes.

According to an embodiment, said temperature is reached within 0.1 to 15 minutes, within at least 0.5 minute within 1 minute or within 2 minutes, but within less than 12 minutes of less than 10 minutes. According to an embodiment said plant material is kept at the set temperature for a period of between 0.1 to 30 minutes.

According to an embodiment, the temperature of the plant material is reduced by at least 20 degrees Celsius, at least 30 or at least 40 degrees, within less than 30 minutes, less than 20 minutes or less than 10 minutes. Such cooling rate could be essential for reaching the desired product quality.

According to some embodiments, said plant material is not packed during sterilization. According to some embodiments the bulk density of said plant material is between 0.05 and 1 Kg/Liter. According to an embodiment the bulk density is greater than 0.1 greater than 0.15 or greater than 0.2 Kg/Liter. According to an embodiment, it is less than 0.9, less than 0.8, less than 0.7, less than 0.6 or less than 0.5 Kg/Liter.

According to an embodiment, sterilization is carried out by heating. In some such embodiments, heating is applied substantially homogeneously to the material being sterilized i.e. variations between a temperature applied at different points of the material should be no greater than about 5° C.

According to an embodiment, sterilization is carried out on batches of partially dried cannabis plant material.

According to some such embodiments, batches are harvest batches i.e. batches from different harvests, which may comprise different strains of cannabis plant, having different active components, and/or concentrations of components. According to some embodiments, different harvest batches are from different strains of cannabis plant. According to some such embodiments, the room or chamber in which the sterilization is conducted is itself sterilized between sterilization of successive batches.

In some such embodiments, the batches each have a weight of from about 1 to 50 kg, such as from about 1 to about 20 kg or from about 1 to about 10 kg.

According to an alternative embodiment, sterilization of partially dried cannabis material is carried out as a continuous process.

A person skilled in the art would appreciate that the conditions maintained during sterilizing affect sterilizing efficacy, as well as other properties of the sterilized plant material.

A person skilled in the art would also appreciate the conflict between providing sufficient energy for achieving the required sterilizing efficacy and minimizing the degradation of cannabis active components.

Sterilizing conditions to be controlled include temperature profile (including the treatment set temperature, the rate of heating to said set temperature and the rate of cooling), length of time at the set temperature, the relative humidity to which the treated plant material is exposed, the packing of the treated plant material and the setup of the sterilizing source, e.g. the setup of the irradiation source.

According to an embodiment, said cannabis plant material is maintained in an oxygen deficient environment during substantially an entirety of the sterilizing duration. According to some embodiments, a rapid cooling phase is required, which may be achieved, for example, by exposure of the plant material to ambient air.

As used herein, the term “oxygen deficient environment” is intended to refer to an environment in which the oxygen content is at least 25% less than the oxygen content of about 20% which is present in the atmosphere under natural conditions i.e. an oxygen deficient atmosphere has an oxygen content of less than 15%, less than 10%, less than 5% or even less than 2%.

According to some embodiments, said oxygen deficient environment is an ambient environment enriched with nitrogen. As used herein, the phrase “enriched with nitrogen” is intended to refer to an environment having a higher nitrogen content than that present in ambient air under natural conditions. Hence, an environment enriched with nitrogen may have a nitrogen concentration of greater than 80%, greater than 85%, greater than 90%, greater than 95% or even greater than 98% nitrogen.

According to an embodiment, said sterilizing is carried out in a sterilizing chamber, wherein said plant material is loaded into said chamber and said chamber is nitrogen flashed prior to carrying out said sterilization, e.g. prior to or simultaneously with temperature elevation to the set heat point. According to an embodiment, a nitrogen and moisture enriched atmosphere is circulated through said chamber during said sterilizing.

According to an embodiment, said sterilizing conditions (including temperature profile and relative humidity) are selected so that the water content of the sterilized plant material does not change during sterilization, or undergoes minimal change, so that M3 is approximately equal to M1, e.g. within 0.5% wt, within 1% wt, within 2% wt, with 3% wt or within 4% wt. Thus for example, the water content of the partially dried plant material is 12% wt and that of the sterilized material is 11-13% wt, 11-14% wt, 10-13% wt, 10-14% wt, etc.

According to an alternative embodiment, the water content of the treated cannabis material changes during said sterilizing, e.g. during sterilizing.

According to an embodiment, M3 differs from M1 by no more than about ±5%, such as ±about 4%, about 4%, about 3%, about 1%, about 1%, or about 0.5%. According to an embodiment, M3=M1.

According to various embodiments, drying of the cannabis plant material is required, typically to provide a water content in the range of from about 8% wt to about 16% wt. Such drying is required, e.g. for smoking the cannabis where excess moisture interferes with proper utilization. Too low a water content is also problematic. Similar requirements exist also for cannabis utilization via vaporizers. Water content control is even required for preparing the cannabis for extraction.

According to an embodiment, when M3 is at least about 16% wt, adjusting M3 according to step (iv) comprises further drying said sterilized partially dried cannabis plant material to provide said water content of M2. According to some such embodiments, said further drying said partially dried cannabis plant material having water content of M3 is carried out simultaneously with said sterilizing said partially dried cannabis plant, to form sterilized mostly dried cannabis plant material having a water content of M2.

According to some embodiments, the partially dried cannabis material is further dried as a result of heating which occurs during the sterilization step such that the water content of the sterilized partially dried cannabis plant material is no greater than 16%.

According to an embodiment, when said M3 is less than about 8% wt, said adjusting said M3 according to step (iv) comprises adding moisture to provide said sterilized partially dried cannabis plant material having a water content of M2 wherein M2 is at least about 8% wt.

According to an embodiment said adding moisture is carried out by maintaining in an environment with sufficiently high humidity, e.g. in the range between 30-70%, 30-60%, 40-70% or 40-60%, such as 40%, 45%, 50%, 55% or 60%.

According to another embodiment, the cannabis plant material is dried prior to said sterilization to a desired water content, e.g. 8-16% wt in order to reach the desired drying and reactions profile. According to an embodiment, moisture loss may take place during said sterilizing. According to an embodiment, water is added to said plant material prior to said sterilizing. According to another embodiment, said water addition prior to said sterilizing is adjusted so that the water content post sterilizing (and post water lost on sterilizing) reaches a desired level of 8-16% wt.

According to an embodiment, further drying said sterilized partially dried cannabis plant material having water content M2 is carried out by heating at a temperature in the range of from about 10° C. to about 70° C., from about 15° C. to about 45° C. or from about 20° C. to about 40° C., such as about 10° C., about 15° C., about 20° C., about 25° C., about 30° C., about 35° C., about 40° C., about 45° C., about 50° C., about 55° C., about 60° C., about 65° C. or about 70° C.

According to an embodiment, said further drying said sterilized partially dried cannabis plant material is carried out for a duration of between 1 and 15 days, such as for 1 day, 2 days, 3 days, 4 days, 5 days, 6 days, 7 days, 8 days, 9 days, 10 days, 11 days, 12 days, 13 days, 14 days or 15 days.

According to an aspect of some embodiments of the invention, there is provided a method for cannabis drying comprising:
(i) providing a system comprising at least one primary drying chamber, which at least one primary drying chamber has a volume V1, and in operation provides a relative humidity RH1 and a temperature T1; and at least one secondary drying chamber which at least one secondary drying chamber has a volume V2, and in operation provides a relative humidity RH2 and a temperature T2, and wherein said system comprises at least one selected from the group consisting of
   (a) said V2 being greater than said V1;
   (b) said RH2 being lower than said RH1; and
   (c) said T2 being higher than said T1;
(ii) providing a feed cannabis plant material having a water content of M0 percent and a relative water load L0 wherein L0 equals M0/(100−M0) and wherein M0 is greater than 45%;
(iii) keeping said feed cannabis plant material in said at least one primary drying chamber for a duration of D1 days, during at least a portion of which said duration of D1 days said primary drying chamber is in operation, whereby said water content Mₒ of said feed cannabis plant material drops to form a partially dried cannabis plant material having a water content of M1 percent and a relative water load L1 wherein L1 equals M1/(100−M1);
(iv) transferring said partially dried cannabis plant material into said at least one secondary drying chamber and keeping it there for a duration of D2 days, during at least a portion of which said duration of D2 days said drying chamber is in operation, whereby said water content M1 of said partially dried cannabis plant material drops to form a mostly dried cannabis plant material with water content of M2 percent, and a relative water load L2 wherein L2 equals M2/(100−M2) and wherein said M2 is less than 20%: and optionally
(v) maintaining said mostly dried cannabis plant material for at least 3 days at relative humidity lower than said RH1,
wherein said T1, said T2, said RH1, said RH2, said D1 and said D2 are selected so that (L0−L1)/(L1−L2) is greater than 2.

According to an embodiment, T2 is less than 60° C., less than 55° C., less than 50° C. or less than 40° C.

According to an embodiment, D2/D1 is greater than 2, greater than 3, greater than 4 or greater than 5. According to an embodiment, D2/D1 is no greater than 15.

According to an embodiment, said system comprises at least two selected from the group consisting of (a) said V2 being greater than said V1; (b) said RH2 being lower than said RH1; and (c) said T2 being higher than said T1.

According to an embodiment, said system comprises each of (a) said V2 being greater than said V1; (b) said RH2 being lower than said RH1; and (c) said T2 being higher than said T1.

According to an embodiment, said V2 is greater than said V1 by at least 50%, at least 100%, at least 150%, or at least 200%.

According to an embodiment, said RH2 is lower than said RH1 by at least 5%, at least 10%, at least 15% or at least 20%.

According to an embodiment, said T2 is higher than said T1 by at least 2° C., at least 4° C., at least 6° C., at least 8° C. or at least 10° C.

According to an embodiment, M2 is less than 18, less than 16, less than 14, less than 13, less than 12, less than 11 or less than 10.

According to an embodiment, M2 is greater than 2, greater than 4, greater than 6 or greater than 8.

According to an embodiment, (L0−L1)/(L1−L2) is greater than 3, greater than 4, greater than 5, greater than 6, greater than 7, greater than 8, greater than 9 or greater than 10.

According to an embodiment, the method further comprises circulating air in said at least one primary drying chamber at C1 volumes per hour and optionally circulating air in said at least one secondary drying chamber at C2 volumes per hour, wherein said C1 is greater than said C2 by at least 50%, at least 60%, at least 70%, at least 80%, at least 90% or at least 100%.

According to an embodiment, the method further comprises sterilizing said feed cannabis plant material after Si days from said time of said providing said feed cannabis plant material, wherein said Si is in the range between D1-1 and D1+5, such as D1-1, D1, D1+1, D1+2, D1+3, D1+4 or D1+5.

According to an embodiment, sterilizing comprises exposing to at least one of gamma irradiation, beta irradiation, ultraviolet irradiation, microwave irradiation, radio-frequency irradiation, a contact with an oxidant and plasma treatment or combinations thereof.

According to an embodiment, at least one primary drying chamber received unsterilized cannabis plant material, such that the first primary drying chamber acts as a ‘quarantine’ chamber for potentially contaminated cannabis plant material prior to sterilization.

According to an embodiment, the feed cannabis plant material is tested for microbiological contamination prior to and subsequent to said sterilizing.

According to an embodiment, sterilizing is carried out for a sterilizing duration D3, and at energy input E, wherein said D3 and E are selected to provide a microbiological count following said D3 of less than 20,000 colony-forming units per gram.

According to an embodiment, said at least one primary drying chamber comprises multiple primary drying chambers. wherein said volume V1 is a sum of the volumes of each of said multiple primary drying chambers, said temperature T1 is an average of the temperature of each of said multiple primary drying chambers and said relative humidity RH1 is an average of the relative humidity of each of said multiple primary drying chambers.

According to an embodiment, said multiple primary drying chambers comprise two, three, four, five, six or more primary drying chambers.

According to an embodiment, a variation in temperature between each of said multiple primary drying chambers is less than 20° C., less than 15° C., less than 10° C. or less than 5° C.

According to an embodiment, a variation in relative humidity between each of said multiple primary drying chambers is less than 20%, less than 15%, less than 10% or less than 5%.

According to an embodiment, said at least one secondary drying chamber comprises multiple secondary drying chambers. wherein said volume V2 is a sum of the volumes of each of said multiple secondary drying chambers, said temperature T2 is an average of a temperature of each of said multiple secondary drying chambers and said relative humidity RH2 is an average of a relative humidity of each of said multiple secondary drying chambers.

According to an embodiment, said multiple secondary drying chambers comprise two, three, four, five, six, eight, ten or more primary drying chambers.

According to an embodiment, the number of said multiple secondary drying chambers is at least twice the number of said primary drying chambers.

According to an embodiment, a variation in said temperature between each of said multiple secondary drying chambers is less than 20° C., less than 15° C., less than 10° C. or less than 5° C.

According to an embodiment, a variation in said relative humidity between each of said multiple secondary drying chambers is less than 20%, less than 15%, less than 10% or less than 5%.

According to an embodiment, said feed cannabis plant material comprises cannabis flowers.

According to an embodiment, the method further comprises at least one selected from the group consisting of a) determining said water content M0 of said feed cannabis material prior to said keeping said feed cannabis plant material in said at least one primary drying chamber, b) determining said water content M1 of said partially dried cannabis plant material prior to said keeping said partially dried cannabis material in said at least one secondary drying chamber, and c) determining said water content M2 of said mostly dried cannabis plant material subsequent to said keeping said partially dried cannabis plant material in said secondary drying chamber for a duration of D2 days.

According to an embodiment, said determining water content comprises a method selected from loss on drying and Near Infra Red measurements. According to an embodiment, the feed cannabis plant material is spread on at least one tray in the at least one primary drying chamber and/or the partially dried cannabis plant material is spread on at least one tray in the at least one secondary drying chamber.

According to an embodiment, the method further comprises packing said mostly dried cannabis plant material, wherein said packing is optionally carried out subsequent to step (iv).

According to an aspect of some embodiments of the present invention, there is further provided a system for cannabis drying, comprising (i) at least one primary drying chamber, having a volume V1, and configured to provide, during operation, a relative humidity RH1 and temperature T1; and (ii) at least one secondary drying chamber, having a volume V2, and configured to provide, during operation, a relative humidity RH2 and temperature T2, wherein said system is characterized by at least one selected from the group consisting of (a) said V2 being greater than said V1; (b) said RH2 being lower than said RH1; and (c) said T2 being higher than said T1.

According to an embodiment, said system comprises at least two selected from the group consisting of (a) said V2 being greater than said V1; (b) said RH2 being lower than said RH1; and (c) said T2 being higher than said T1.

According to an embodiment, said system comprises each of (a) said V2 being greater than said V1; (b) said RH2 being lower than said RH1; and (c) said T2 being higher than said T1.

According to an embodiment, said V2 is greater than said V1 by at least 50%, at least 100%, at least 150%, or at least 200%.

According to an embodiment, said RH2 is lower than said RH1 by at least 5%, at least 10%, at least 15% or at least 20%.

According to an embodiment, said T2 is higher than said T1 by at least 2° C., at least 4° C., at least 6° C., at least 8° C. or at least 10° C.

According to an embodiment, the system further comprises a sterilizing unit.

According to an embodiment, T2 is less than 60° C., less than 55° C., less than 50° C. or less than 40° C.

According to an embodiment, the system is configured to circulate air in said at least one primary drying chamber of C1 volumes per hour and optionally circulate air in said at least one secondary drying chamber of C2 volumes per hour, wherein said C1 is greater than said C2 by at least 50%, at least 60%, at least 70%, at least 80%, at least 90% or at least 100%

According to an embodiment, said sterilizing unit is configured to provide at least one of gamma irradiation, beta irradiation, ultraviolet irradiation, microwave irradiation, radio-frequency irradiation, a contact with an oxidant and plasma treatment.

According to an embodiment, said sterilizing unit is configured to operate at a sub-atmospheric pressure.

According to an embodiment, said at least one primary drying chamber comprises multiple primary drying chambers. wherein said volume V1 is a sum of said volumes of said multiple primary drying chambers, said temperature T1 is the average temperature of said multiple primary drying chambers and said relative humidity RH1 is the average relative humidity of said multiple primary drying chambers.

According to an embodiment, said multiple primary drying chambers comprise two, three, four, five, six or more primary drying chambers.

According to an embodiment, a variation in temperature between each of said multiple primary drying chambers is less than 20° C., less than 15° C., less than 10° C. or less than 5° C.

According to an embodiment, a variation in relative humidity between each of said multiple primary drying chambers is less than 20% m less than 15° C., less than 10° C. or less than 5° C.

According to an embodiment, said at least one secondary drying chamber comprises multiple secondary drying chambers. wherein said volume V2 is a sum of said volumes of each of said multiple secondary drying chambers, said temperature T2 is an average of said temperature of each of said multiple secondary drying chambers and said relative humidity RH2 is an average of said relative humidity of each of said multiple secondary drying chambers.

According to an embodiment, said multiple secondary drying chambers comprise two, three, four, five, six, eight, ten or more primary drying chambers.

According to an embodiment, the number of said multiple secondary drying chambers is at least twice the number of said primary drying chambers.

According to an embodiment, a variation in temperature between each of said multiple secondary drying chambers is less than 20° C., less than 15%, less than 10% or less than 5%.

According to an embodiment, a variation in relative humidity between each of said multiple secondary drying chambers is less than 20%, less than 15%, less than 10% or less than 5%.

According to an embodiment, said system comprises at least two selected from the group consisting of (a) said V2 being greater than said V1; (b) said RH2 being lower than said RH1; and (c) said T2 being higher than said T1.

According to an embodiment, said system comprises each of (a) said V2 being greater than said V1; (b) said RH2 being lower than said RH1; and (c) said T2 being higher than said T1.

The scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the claimed embodiments.

### EXAMPLES

### Example 1

A system is provided comprising four primary drying chambers and eight secondary drying chambers. The total combined volume of the primary drying chambers is 100 meters³ and the total combined volume of the secondary drying chambers is 200 meters³.

During operation, the average relative humidity of the primary drying chambers is 65% and the relative humidity of the secondary drying chambers is 55%. During operation, the average temperature of the primary drying chambers is 20° C. and the average temperature of the primary drying chambers is 26° C. The feed cannabis plant material has a water content (M0) of 65%. The feed cannabis plant material is dried for a duration (D1) of 2 days to form a partially dried cannabis plant material having a water content (M1) of 30%. The partially dried cannabis plant material is dried for a duration (D2) of 8 days to form a mostly dried cannabis plant material having a water content (M2) of 11%.

Four days after providing the feed cannabis plant material, the mostly dried cannabis plant material is sterilized for a duration (D3) of 10 minutes.

### Example 2

A system is provided comprising six primary drying chambers and twelve secondary drying chambers. The total combined volume of the primary drying chambers is 300 meters³ and the total combined volume of the secondary drying chambers is 1200 meters³.

During operation, the average relative humidity of the primary drying chambers is 62% and the relative humidity of the secondary drying chambers is 58%. During operation, the average temperature of the primary drying chambers is 24° C. and the average temperature of the primary drying chambers is 28° C. The feed cannabis plant material has a water content (M0) of 62%. The feed cannabis plant material is dried for a duration (D1) of 3 days to form a partially dried cannabis plant material having a water content (M1) of 25%. The partially dried cannabis plant material is dried for a duration (D2) of 10 days to for a mostly dried cannabis plant material having a water content (M2) of 11.5%.

Eight days after providing the feed cannabis plant material, the mostly dried cannabis plant material is sterilized for a duration (D3) of 25 minutes.

1. A method for drying and sterilizing of cannabis plant material, the method comprising:
(i) providing feed cannabis plant material having a water content of M0, wherein M0 is greater than 45%;
(ii) partially drying said feed cannabis plant material to form a partially dried cannabis plant material having a water content of M1, wherein M1 is in the range of between 12% wt and 40% wt;
(iii) sterilizing said partially dried cannabis plant material to form sterilized partially dried cannabis plant material having a water content of M3; and
(iv) when said M3 is less than 8% wt or greater than 16% wt, adjusting said M3 to provide a water content of M2, wherein said M2 is between 8% wt and 16% wt.

2. The method of claim 1, wherein steps (ii) and (iii), and optionally step (iv), when required, are carried out at a single processing site.

3. The method of claim 1, wherein said partially dried cannabis plant material is maintained in an oxygen deficient environment during at least a fraction of a duration of said sterilizing.

4. The method of claim 1, wherein said M3 differs from M1 by no more than ±5%.

5. The method of claim 1, wherein when M3 is at least 16% wt, said adjusting M3 according to step (iv) comprises further drying said sterilized partially dried cannabis plant material to provide said water content of M2 and when said M3 is less than 8% wt, said adjusting said M3 according to step (iv) comprises adding moisture to provide said sterilized partially dried cannabis plant material having a water content of M2.

6. (canceled)

7. The method of claim 5, wherein said further drying said partially dried cannabis plant material having water content M3 is carried out simultaneously with said sterilizing said partially dried cannabis plant material.

8. The method of claim 1, wherein said feed cannabis plant material comprises cannabis flowers.

9. (canceled)

10. The method of claim 1, wherein said partially drying said feed cannabis plant material is carried out for a duration of between 0.5 and 5 days.

11. The method of claim 1, wherein said sterilizing is carried out by a method selected from the group consisting of gamma irradiation, beta irradiation, ultraviolet irradiation, microwave irradiation, radio-frequency irradiation and plasma treatment and combinations thereof.

12. The method of claim 1, wherein said sterilizing is carried out by applying radio-frequency irradiation, bringing the temperature of said plant material to a temperature in the range of from about 60 to about 90 degrees Celsius.

13. (canceled)

14. The method of claim 1, wherein said further drying said sterilized partially dried cannabis plant material is carried out for a duration of between 1 and 15 days.

15. A method for drying of cannabis plant material, the method comprising:
(i) providing a system comprising at least one primary drying chamber, which at least one primary drying chamber has a volume V1, and in operation provides a relative humidity RH1 and a temperature T1; and at least one secondary drying chamber which at least one secondary drying chamber has a volume V2, and in operation provides a relative humidity RH2 and a temperature T2, and wherein said system comprises at least one selected from the group consisting of
(a) said V2 being greater than said V1;
(b) said RH2 being lower than said RH1; and
(c) said T2 being higher than said T1;
(ii) providing a feed cannabis plant material having a water content of M0 percent and a relative water load L0 wherein L0 equals M0/(100−M0) and wherein M0 is greater than 45%;
(iii) keeping said feed cannabis plant material in said at least one primary drying chamber for a duration of D1 days, during at least a portion of which said duration of D1 days said primary drying chamber is in operation, whereby said water content M0 of said feed cannabis plant material drops to form a partially dried cannabis plant material having a water content of M1 percent and a relative water load L1 wherein L1 equals M1/(100−M1);
(iv) transferring said partially dried cannabis plant material into said at least one secondary drying chamber and keeping it there for a duration of D2 days, during at least a portion of which said duration of D2 days said drying chamber is in operation, whereby said water content M1 of said partially dried cannabis plant material drops to form a mostly dried cannabis plant material with water content of M2 percent, and a relative water load L2 wherein L2 equals M2/(100−M2) and wherein said M2 is less than 20%: and optionally
(v) maintaining said mostly dried cannabis plant material for at least 3 days at relative humidity lower than said RH1,
wherein said T1, said T2, said RH1, said RH2, said D1 and said D2 are selected so that (L0−L1)/(L1−L2) is greater than 2.

16. The method of claim 15, wherein said T2 is less than 60 degrees Celsius and/or wherein D2/D1 is greater than 2.

17. (canceled)

18. The method of claim 15, further comprising circulating air in said at least one primary drying chamber at C1 volumes per hour and optionally circulating air in said at least one secondary drying chamber at C2 volumes per hour, wherein said C1 is greater than said C2 by at least 50%.

19. The method of claim 15, further comprising sterilizing said feed cannabis plant material after Si days from said time of said providing said feed cannabis plant material, wherein said Si is in the range between D1-1 and D1+5.

20. The method of claim 19, wherein said sterilizing comprises exposing to at least one of gamma irradiation, beta irradiation, ultraviolet irradiation microwave irradiation, radio-frequency irradiation, a contact with an oxidant, and plasma treatment.

21. (canceled)

22. The method of claim 20, wherein said sterilizing is carried out for a sterilizing duration D3, and at energy input E, wherein said D3 and E are selected to provide a microbiological count following said D3 of less than 20,000 colony-forming units per gram.

23-28. (canceled)

29. The method of claim 15, wherein said feed cannabis plant material comprises cannabis flowers.

30-36. (canceled)

37. A system for drying of cannabis plant material, the system comprising:
(i) at least one primary drying chamber, having a volume V1, and configured to provide, during operation, a relative humidity RH1 and temperature T1; and
(ii) at least one secondary drying chamber, having a volume V2, and configured to provide, during operation, a relative humidity RH2 and temperature T2,
wherein said system is characterized by at least one selected from the group consisting of (a) said V2 being greater than said V1; (b) said RH2 being lower than said RH1; and (c) said T2 being higher than said T1.

38. The system of claim 37, further comprising a sterilizing unit.

39-53. (canceled)
